SEQUENCE LISTING (32 sequences)

SEQ ID NO: 1 - protein, 107 aa, Artificial
  recombinant antibody variable region
DIQMTQSPSSLSASVGDRVTITCRASQSISNNLHWYQQKPGKAPKLLIKYVFQSISGVPSRFSGSGSGTDFTFTISSLQPEDFATYYCQQSNSWPLTFGGGTKVEIK

SEQ ID NO: 2 - protein, 107 aa, Artificial
  recombinant antibody variable region
DIQMTQSPSSLSASVGDRVTITCRASQSISNNLHWYQQKPGKAPKLLIKYAFQSISGVPSRFSGSGSGTDFTFTISSLQPEDFATYYCQQANSWPLTFGGGTKVEIK

SEQ ID NO: 3 - protein, 107 aa, Artificial; identical to SEQ ID NO: 2
  recombinant antibody variable region

SEQ ID NO: 4 - protein, 108 aa, Artificial
  recombinant antibody variable region
DIQMTQSPSSLSASVGDRVTITCRASQSISNNLHWYQQKPGKAPKLLIKYTFQSISGVPSRFSGSGSGTDFTFTISSLQPEDFATYYCQQANSWPLTFGGGTKVEIKE

SEQ ID NO: 5 - protein, 107 aa, Artificial
  recombinant antibody variable region
DIQMTQSPSSLSASVGDRVTITCRASQSISNNLHWYQQKPGKAPKLLIKYAFQSISGVPSRFSGSGSGTDFTFTISSLQPEDFATYYCQQSNSWPLTFGGGTKVEIK

SEQ ID NO: 6 - protein, 107 aa, Artificial; identical to SEQ ID NO: 5
  recombinant antibody variable region

SEQ ID NO: 7 - protein, 107 aa, Artificial
  recombinant antibody variable region
EIVLTQSPATLSLSPGERATLSCQASQSISNFLHWYQQRPGQAPRLLIRYRSQSISGIPARFSGSGSGTDFTLTISSLEPEDFAVYYCQQSGSWPLTFGGGTKVEIK

SEQ ID NO: 8 - protein, 112 aa, Artificial
  recombinant antibody variable region
DVVMTQTPLTLSVTIGQPASISCKSSQSLLYSNGKTYLNWLLQRPGQSPKRLIYLVSKLDSGVPDRFTGSGSGTDFTLKISRVEAEDLGVYYCVQGSHFPWTFGGGTKLEIK

SEQ ID NO: 9 - protein, 120 aa, Artificial
  recombinant antibody variable region
QMQLVQSGPEVKKPGTSVKVSCKASGFTFTDYSMNWVRQARGQRLEWIGFIRNKANDYTTEYSASVKGRVTITRDMSTSTAYMELSSLRSEDTAVYYCARYPRYHAMDSWGQGTSVTVSS

SEQ ID NO: 10 - protein, 120 aa, Artificial
  recombinant antibody variable region
QMQLVQSGPEVKKPGTSVKVSCKASGFTFDDYSMTWVRQARGQRLEWIGFIRNKANAYTTEYSASVKGRVTITRDMSTSTAYMELSSLRSEDTAVYYCARYPRYHAMDSWGQGTSVTVSS

SEQ ID NO: 11 - protein, 120 aa, Artificial
  recombinant antibody variable region
QMQLVQSGPEVKKPGTSVKVSCKASGFTFDDYSMNWVRQARGQRLEWIGFIRNKANDYTTEYSASVKGRVTITRDMSTSTAYMELSSLRSEDTAVYYCARYPRHHAMDSWGQGTSVTVSS

SEQ ID NO: 12 - protein, 120 aa, Artificial
  recombinant antibody variable region
QMQLVQSGPEVKKPGTSVKVSCKASGFTFDDYSMTWVRQARGQRLEWIGFIRNKANDYTTEYSASVKGRVTITRDMSTSTAYMELSSLRSEDTAVYYCARYPRYHAMDSWGQGTSVTVSS

SEQ ID NO: 13 - protein, 120 aa, Artificial
  recombinant antibody variable region
QMQLVQSGPEVKKPGTSVKVSCKASGFTFDDYSMTWVRQARGQRLEWIGFIRNKANAYTTEYSASVKGRVTITRDMSTSTAYMELSSLRSEDTAVYYCARYPRHHAMDSWGQGTSVTVSS

SEQ ID NO: 14 - protein, 120 aa, Artificial
  recombinant antibody variable region
QMQLVQSGPEVKKPGTSVKVSCKASGFTFTDYSMNWVRQARGQRLEWIGFIRNKANSYTTEYSASVKGRVTITRDMSTSTAYMELSSLRSEDTAVYYCARYPRHHAMDSWGQGTSVTVSS

SEQ ID NO: 15 - protein, 117 aa, Artificial
  recombinant antibody variable region
QVQLVESGGGVVQPGRSLRLSCAASGFTFSSYDMSWVRQAPGKGLEWVAKVSSGGGSTYYLDTVQGRFTISRDNSKNTLYLQMNSLRAEDTAVYYCARHLHGSFASWGQGTTVTVSS

SEQ ID NO: 16 - protein, 115 aa, Artificial
  recombinant antibody variable region
EVQLQQSGPELVKTGASVKISCKASGYSFTGYYMHWVKQSHGKSLEWIGYISCYNGVTSYNQKFKGKATFTVDTSSSTAYMQFNSLTSEDSAVYYCARSHAMDYWGQGTSVTVSS

SEQ ID NO: 17 - DNA, 44 bp, Artificial
  primer
acacaacagagtacgctgactctgtgaagggtagagtcaccatt

SEQ ID NO: 18 - DNA, 48 bp, Artificial
  Primer
ggtggtggtagcacctactatgcagacactgtgcagggccgattcacc

SEQ ID NO: 19 - DNA, 48 bp, Artificial
  Primer
ggtgaatcggccctgcacagtgtctgcatagtaggtgctaccaccacc

SEQ ID NO: 20 - DNA, 49 bp, Artificial
  primer
gttacaatggtgttactagctacgccgacaagttcaagggcaaggccac

SEQ ID NO: 21 - DNA, 49 bp, Artificial
  Primer
gtggccttgcccttgaacttgtcggcgtagctagtaacaccattgtaac

SEQ ID NO: 22 - DNA, 43 bp, Artificial
  Primer
ctacatgcactgggtcaagcaggcccatggaaagagccttgag

SEQ ID NO: 23 - DNA, 43 bp, Artificial
  Primer
ctcaaggctctttccatgggcctgcttgacccagtgcatgtag

SEQ ID NO: 24 - protein, 120 aa, Artificial
  recombinant antibody variable region
QMQLVQSGPEVKKPGTSVKVSCKASGFTFTDYSMNWVRQARGQRLEWIGFIRNKANDYTTEYADSVKGRVTITRDMSTSTAYMELSSLRSEDTAVYYCARYPRYHAMDSWGQGTSVTVSS

SEQ ID NO: 25 - protein, 120 aa, Artificial
  recombinant antibody variable region
QMQLVQSGPEVKKPGTSVKVSCKASGFTFDDYSMTWVRQARGQRLEWIGFIRNKANAYTTEYADSVKGRVTITRDMSTSTAYMELSSLRSEDTAVYYCARYPRYHAMDSWGQGTSVTVSS

SEQ ID NO: 26 - protein, 120 aa, Artificial
  recombinant antibody variable region
QMQLVQSGPEVKKPGTSVKVSCKASGFTFDDYSMNWVRQARGQRLEWIGFIRNKANDYTTEYADSVKGRVTITRDMSTSTAYMELSSLRSEDTAVYYCARYPRHHAMDSWGQGTSVTVSS

SEQ ID NO: 27 - protein, 120 aa, Artificial
  recombinant antibody variable region
QMQLVQSGPEVKKPGTSVKVSCKASGFTFDDYSMTWVRQARGQRLEWIGFIRNKANDYTTEYADSVKGRVTITRDMSTSTAYMELSSLRSEDTAVYYCARYPRYHAMDSWGQGTSVTVSS

SEQ ID NO: 28 - protein, 120 aa, Artificial
  recombinant antibody variable region
QMQLVQSGPEVKKPGTSVKVSCKASGFTFDDYSMTWVRQARGQRLEWIGFIRNKANAYTTEYADSVKGRVTITRDMSTSTAYMELSSLRSEDTAVYYCARYPRHHAMDSWGQGTSVTVSS

SEQ ID NO: 29 - protein, 120 aa, Artificial
  recombinant antibody variable region
QMQLVQSGPEVKKPGTSVKVSCKASGFTFTDYSMNWVRQARGQRLEWIGFIRNKANSYTTEYADSVKGRVTITRDMSTSTAYMELSSLRSEDTAVYYCARYPRHHAMDSWGQGTSVTVSS

SEQ ID NO: 30 - protein, 117 aa, Artificial
  recombinant antibody variable region
QVQLVESGGGVVQPGRSLRLSCAASGFTFSSYDMSWVRQAPGKGLEWVAKVSSGGGSTYYADTVQGRFTISRDNSKNTLYLQMNSLRAEDTAVYYCARHLHGSFASWGQGTTVTVSS

SEQ ID NO: 31 - protein, 115 aa, Artificial
  recombinant antibody variable region
EVQLQQSGPELVKTGASVKISCKASGYSFTGYYMHWVKQSHGKSLEWIGYISCYNGVTSYADKFKGKATFTVDTSSSTAYMQFNSLTSEDSAVYYCARSHAMDYWGQGTSVTVSS

SEQ ID NO: 32 - protein, 115 aa, Artificial
  recombinant antibody variable region
EVQLQQSGPELVKTGASVKISCKASGYSFTGYYMHWVKQAHGKSLEWIGYISCYNGVTSYADKFKGKATFTVDTSSSTAYMQFNSLTSEDSAVYYCARSHAMDYWGQGTSVTVSS

(19) United States
(12) Patent Application Publication
(10) Pub. No.: US 2006/0019342 A1
(43) Pub. Date: Jan. 26, 2006

(54) Increasing the production of recombinant antibodies in mammalian cells by site-directed mutagenesis

(75) Inventors: William Dall'Acqua, Gaithersburg, MD (US); Herren Wu, Boyds, MD (US); Melissa Damschroder, Germantown, MD (US)

Correspondence Address: JOHNATHAN KLEIN-EVANS, ONE MEDIMMUNE WAY, GAITHERSBURG, MD, 20878, US

(73) Assignee: MEDIMMUNE, INC., Gaithersburg, MD (US)

(21) Appl. No.: 11/165,023

(22) Filed: Jun. 24, 2005

Related U.S. Application Data
(60) Provisional application No. 60/583,184, filed on Jun. 25, 2004.
(60) Provisional application No. 60/624,153, filed on Nov. 2, 2004.

(51) Int. Cl. C07H 21/04; C12P 21/06; C12N 5/06; C07K 16/44

(52) U.S. Cl. 435/69.1; 435/328; 435/320.1; 530/387.3; 536/23.53

(57) ABSTRACT

The present invention relates to a reliable, reproducible method for improving the producibility of an antibody. More specifically, this invention provides a method for modifying the heavy chain of an antibody to improve its producibility in eukaryotic cells. Additionally, the method of the invention may improve both antibody producibility and one or more antigen binding characteristics. The invention further provides modified antibodies which are better produced and which have either no change in their antigen binding characteristics or exhibit improved antigen binding characteristics.

### CROSS REFERENCE TO RELATED APPLICATIONS

This application claims the benefit under 35 U.S.C. §119(e) of the following U.S. Provisional Application Nos. 60/583,184, filed Jun. 25, 2004 and 60/624,153, filed Nov. 2, 2004. The priority applications are hereby incorporated by reference herein in their entirety for all purposes.

### BACKGROUND OF THE INVENTION

The use of antibodies to block the activity of foreign and/or endogenous polypeptides provides an effective and selective strategy for treating the underlying cause of disease. In particular is the use of recombinant monoclonal antibodies (MAb) and antibody fragments as effective therapeutics such as the FDA approved Synagis (Saez-Llorens, X. E., et al., 1998, Pediat. Infect. Dis. J. 17:787-91), an anti-respiratory syncytial virus MAb produced by Medimmune; ReoPro (Glaser, V., 1996, Nat. Biotechnol. 14:1216-17), an anti-platelet Fab antibody fragment from Centocor; and Herceptin (Weiner, L. M., 1999, Semin. Oncol. 26:43-51), an anti-Her2/neu MAb from Genentech.

Standard methods for generating MAbs against candidate protein targets are known by those skilled in the art. Briefly, rodents such as mice or rats are injected with a purified antigen in the presence of adjuvant to generate an immune response (Shield, C. F., et al., 1996, Am. J Kidney Dis. 27:855-64). Rodents with positive immune sera are sacrificed and splenocytes are isolated. Isolated splenocytes are fused to melanomas to produce immortalized cell lines that are then screened for antibody production. Positive lines are isolated and characterized for antibody production. However, the use of rodent MAbs directly as human therapeutic agents may result in the production of the human anti-rodent antibody (HAMA) response (Khazaeli, M. B., et al., 1994, J Immunother. 15:42-52). This response reduces the effectiveness of the antibody by neutralizing the binding activity and by rapidly clearing the antibody from circulation in the body. The HAMA response can also cause significant toxicities with subsequent administrations of rodent antibodies.

In order to reduce the HAMA response the production of human-murine chimeric antibodies in which the genes encoding the mouse heavy and light chain variable regions have been coupled to the genes for human heavy and light chain constant regions to produce chimeric or hybrid antibodies is commonly utilized. In some cases, mouse CDRs have been grafted onto human constant and framework regions with some of the mouse framework amino acids being substituted for correspondingly positioned human amino acids to provide a “humanized” antibody. Examples detailing the production of chimeric and/or humanized antibodies can be found in Jordan et al. U.S. Pat. No. 6,652,863; Winter et al. U.S. Pat. No. 5,225,539; Queen et al. U.S. Pat. Nos. 5,693,761 and 5,693,762; and Adair et al. U.S. Pat. No. 5,859,205, which are incorporated herein by reference in their entirety

Human antibodies can also be generated and “matured” by screening phage display antibody libraries derived from human immunoglobulin sequences. Techniques and protocols required to generate, propagate, screen (pan), and use the antibody fragments from such libraries have recently been compiled (See, e.g., Barbas et al., 2001, Phage Display: A Laboratory Manual, Cold Spring Harbor Laboratory Press and Kay et al. (eds.), 1996, Phage Display of Peptides and Proteins: A Laboratory Manual, Academic Press, Inc., also see, Winter et al. U.S. Pat. No. 6,225,447 and Knappik et al. U.S. Pat. No. 6,300,064; Kufer et al. PCT publication WO 98/46645; Barbas et al. U.S. Pat. No. 6,096,551; and Kang et al. U.S. Pat. No. 6,468,738 each of which is incorporated herein by reference in its entirety.) Typically, phage-displayed antibody fragments are scFv fragments or Fab fragments; when desired, full length antibodies can be produced by cloning the variable regions from the displaying phage into a complete antibody and expressing the full length antibody in a prokaryotic or a eukaryotic host cell.

As glycoproteins, antibodies typically include oligosaccharide (carbohydrate) chains attached to the protein at specific amino acid residues. The number, type, and location of the carbohydrate attachments on the protein can affect key properties of commercial biopharmaceuticals including clearance rate, immunogenicity, biological specific activity, solubility and stability against proteolysis. Humans will typically accept only those biotherapeutics that have particular types of carbohydrate attachments and will often reject glycoproteins that include non-mammalian oligosaccharide attachments. As a result, eukaryotic cells such as yeast and mammalian cell lines (e.g., Chinese Hamster Ovary (CHO), Baby Hamster Kidney (BHK), Human Embryonic Kidney-293 (HEK-293)) are used for the production of the vast majority of these glycoprotein therapeutics because of their capacity to generate glycoforms and perform other post-translational processing patterns that are accepted by human patients. Unfortunately, production of biotherapeutics in mammalian cells can be expensive due to the need to grow these cells in costly cell culture environments and because mammalian cells often produce the proteins in low yields. Thus, expression and production of the engineered antibody is the next hurdle that must be over come for manufacturing of the molecule for clinical materials.

Methods for producing a larger amount of monoclonal antibodies by manipulating the culture conditions have been reported. For example, McCormack et al. (1988, Cell immunol. 115:325-33) reported that antibody production increases when human antibody-producing hybridomas are cultured in an interleukin 2-supplemented culture medium, Grunberg et al. (2003, Biotechniques 34:968-72) demonstrate that the addition of sodium butyrate can increase the expression of recombinant antibody fragments from HEK-293 cells while Knibbs et al. (2003, Biotechnol Prog. 19:9-13) describe the use of hillex microcarrier beads to increase the yield of antibodies from COS-7 cells. However, culture medium based methods such as these do not address the issue of antibody stability and solubility, crucial factors influencing antibody expression and production yields.

Antibody folding efficiency and stability of the antibody fragments often severely limit actual production levels. Thus, it is desirable to increase expression yields by directly engineering the antibody molecule to improve these characteristics. However, the factors influencing antibody stability and expression are still only poorly understood.

Some progress has been made in bacterial systems. For example, Ulrich et al. (1995, Proc. Natl. Acad. Sci. USA 92:11907-11) found that point mutations in the complementarily determining regions (CDRs) can increase the yields of Fab fragments in bacteria. Similarly, Plückthun and colleagues (Knappik et al., 1995, Protein Engng. 8:81-9; Wall et al., 1999, Protein Engng. 12:605-11; Ewert et al., 2003, Biochemistry 42:1517-28 and European. Pat. No. 0938506) showed that primary amino acid sequence can influence folding efficiency and thus production of immunoglobulin (Ig) fragments in E. coli. In addition, Plückthun et al. (European. Pat. No. 0938506 and Ewert et al., 2003, Biochemistry 42:1517-28) disclose a method to improve the solubility and the yield of Ig domains in bacterial systems by making the domain interface more hydrophilic. However, this method is very time consuming. Furthermore, the procedure requires a detailed knowledge and understanding of the 3-dimensional structure of Ig domains and involves the use of expensive computer modeling programs to predict changes that may lead to a stabilized Ig domain.

All of the studies described supra are limited to the expression of Ig fragments and one would not predict that similar point mutations would have a similar effect on folding, stability or expression of an intact antibody. In addition, many of the mutations described fall within the CDRs and could be expected to reduce the affinity or even alter the specificity of an antibody. Furthermore, the studies described supra are limited to expression of immunoglobulin fragments in bacterial systems, specifically E. coli. Human cells and other eukaryotes are subdivided by membranes into many functionally distinct compartments, unlike bacterium, which exist as a single compartment surrounded by a membrane. Eukaryotic cells use “sorting signals,” which are amino acid motifs located within the protein, to target proteins to particular cellular organelles. One type of sorting signal, called a signal sequence, directs proteins destined for the membrane and/or secretion to an organelle called the endoplasmic reticulum (ER). Antibodies fold and assemble after they are directed into the ER aided by a special class of proteins called chaperones (e.g., Hsp70 (BiP), Hsp90 (GRP94) (Melnick et al., 1994, Nature 370:373-5) and Erp72 (Wiest et al., 1990, J Cell Biol. 110:1501-11). In addition, protein disulfide isomerase (PDI) is involved in the generation of the stabilizing disulphide bonds. In contrast, the chaperone content of the periplasmic space of bacterium is far more modest and there is no evidence for ATP in this compartment. In fact, Plückthun indicates that while primary protein structure is the most important factor in E. coli it is the chaperone proteins that are important factors affecting folding, and thus production, in eukaryotes, (see the discussion section of Knappik et al., 1995, Protein Engng. 8:81-9). Thus, one would not expect that protein alterations increasing Ig domain production in bacterial systems (e.g., E. coli) would be applicable to the expression and production of full length antibodies in eukaryotic cells.

Park et al. (U.S. Pat. No. 6,455,677) disclose certain framework modifications of a FAPα-specific antibody, which improve the producibility of this antibody. However, the methodology used was time consuming requiring the screening of numerous mutations as well as light and heavy chain combinations. Additionally, they did not demonstrated that the modifications made would be widely applicable to other antibodies.

Steipe et al. (U.S. Pat. No. 6,262,238) disclose a different approach for antibody stabilization involving amino acid substitutions in the variable domain of the light and/or heavy chains. However, this approach requires the substitution of numerous amino acids without a clear indication of which are important for stabilization of the antibody. Furthermore, alterations of the variable domains of antibodies can have deleterious effects on the binding specificity and/or affinity of the altered antibody. Mutations that alter the binding specificity or reduce the affinity of an antibody may render it clinically and therefore commercially worthless. Thus, this approach involves laborious screening to identify those mutations, which stabilize the antibody without negatively affecting the binding affinity or specificity.

In many instances recombinant expression of native, chimeric and/or CDR-grafted antibodies in mammalian cell culture systems is poor due to improper folding and reduced secretion. Improper folding can lead to poor assembly of heavy and light chains or a transport incompetent conformation that forbids secretion of one or both chains. It is generally accepted that the light chain confers the ability of secretion of the assembled protein in eukaryotic cells, as it is required for the release of the heavy chain from BiP (Lee et al., 1999, Mol Biol Cell., 10:2209-19; Vanhove et al., 2001, Immunity 15:105-14). Given the important role of the light chain in assembly and secretion of antibodies one would not have predicted that substitutions in the heavy chain alone would dramatically increase antibody production in mammalian cells.

While the market for monoclonal antibodies is estimated to grow 30% a year and reach sales of nearly $24 billion by 2010 there is a severe shortage of antibody manufacturing capacity (Garber, 2001, Nat Biotech. 19:184-5). Another serious limitation relating to the commercial use of antibodies is their producibility in large amounts. Many antibodies with therapeutic or commercial potential are not expressed efficiently and cannot be developed due to inherent production limits. The producibility of an antibody is determined by a large number of factors including but not limited to, the host cell used, the growth conditions, the level of gene expression, the stability of the messenger RNA, the stability of the translated antibody protein, protein folding, level of protein aggregation, and the toxicity of the antibody to the host cell. While progress has been made in understanding how some of these factors influence the overall producibility of an antibody, few methods have been developed that lead to a reliable or reproducible increase in producibility of any antibody. Thus, there is a real need for a rapid and reproducible method for increasing the producibility of recombinant antibodies for both clinical development and pharmaceutical manufacturing.

The present invention provides for the first time an antibody engineering method that will reproducibly increase antibody production in eukaryotic cells (e.g., mammalian cell lines) without resulting in a significant negative effect on the binding characteristics of the modified antibody. The method of the present invention eliminates the need for costly and time consuming random mutagenesis techniques that can result in an antibody with altered binding affinity and/or specificity while reliably increasing antibody production from eukaryotic cells.

Citation or discussion of a reference herein shall not be construed as an admission that such is prior art to the present invention.

### SUMMARY OF THE INVENTION

The inventors have made the surprising discovery that specific residues of the immunoglobulin heavy chain play an important role in the producibility (e.g., production levels, yield; expression levels) of antibodies in eukaryotic systems. The inventors have further determined that the substitution of these amino acid residues results in an antibody that is produced at significantly higher levels than the unmodified antibody. Although not intending to be bound by any mechanism of action, the amino acid residue substitutions of the invention may result in an increase in antibody productivity by altering any or all of a number of factors known to affect antibody producibility including but not limited to, the level of gene expression, mRNA turnover and/or translation, antibody stability, antibody folding, antibody secretion, antibody aggregation, and the toxicity of the antibody to the host cell.

Mutations of the CDRs can have an adverse affect on the antigen binding properties of an antibody, however, the inventors have found unexpectedly, that certain substitutions in the CDRs that enhance producibility did not negatively affect antigen binding and could actually enhance the antigen binding properties of the modified antibody. Without wishing to be bound by any particular theory, the amino acid residue substitutions of the invention may result in conformational changes that include, but are not limited to, those that have little or no effect on the antigen binding, those that result in an acceptable decrease in antigen binding, and those that result in an improvement in antigen binding.

Accordingly, the present invention provides a novel method for increasing the producibility of antibodies or antibody fragments and provides novel antibody sequences of the same. Also provided by the present invention are antibodies having at least one amino acid residue substitution, wherein the producibility of said substituted antibody is improved compared to the antibody without said substitution.

The method of the invention involves changes of at least one residue of the heavy chain of an antibody of interest which lead to a significant increase in production and which also may improve the antigen binding characteristics of the antibody.

The present invention provides a method for increasing the producibility of an antibody or antibody fragment comprising the steps of: (a) substituting the amino acid residues at positions 40H, 60H, and 61H, utilizing the numbering system set forth in Kabat, of the antibody of interest with alanine, alanine and aspartic acid, respectively; and (b) cultivating the host cell under conditions where the modified antibody polypeptide is expressed by said host cell.

It is specifically contemplated that one skilled in the art may choose to analyze the nature of the amino acids at positions 40H, 60H and 61H of the antibody of interest prior to making any substitutions at these positions.

In a preferred embodiment, the host cell is eukaryotic including eukaryotic microbes such as yeast. In a more preferred embodiment the host cell is mammalian. Such mammalian host cells include but are not limited to, CHO, BHK, HeLa, COS, MDCK, NIH 3T3, W138, NSO, SP/20 and other lymphocytic cells, and human cells such as PERC6, HEK 293.

In a preferred embodiment, the amino acid residue at positions 40H, 60H and 61H will be substituted as described supra.

In other embodiments, the amino acid residues at position 40H and 60H or 40H and 61H or 60H and 61H will be substituted as described supra.

In still other embodiments, the amino acid residues at position 40H or 60H or 61H will be substituted as described supra.

In a preferred embodiment, the method of the invention will result in an antibody with increased expression levels and/or purification yields from a host cell.

In a more preferred embodiment, the method of the invention will result in an antibody with increased expression levels and/or purification yields without negatively affecting antigen binding characteristics.

In a more preferred embodiment, the method of the invention will result in an antibody with both increased expression levels and/or purification yields and improved antigen binding characteristics.

The present invention also provides new antibody polypeptides having modifications of the heavy chain resulting in improved producibility as compared to the unmodified antibody.

In a preferred embodiment, the antibodies of the invention have improved producibility and little or no reduction in antigen binding. More preferably, the antibodies of the invention have both improved producibility and improved antigen binding characteristics.

In another embodiment, the heavy chain modifications of the antibodies of the invention are to residues 40H, 60H, and 61H. Specifically, positions 40H, 60H, and 61H are substituted, where necessary, by alanine, alanine and aspartic acid, respectively.

### BRIEF DESCRIPTION OF THE FIGURES

FIG. 1 is the amino acid sequence of the variable regions of the light chains (V_{L}) (A) and the heavy (V_{H}) (B) of various antibodies of the invention. Shaded: Positions 40H, 60H and 61H (Kabat numbering); Boxed: CDRs (Kabat definition); Each sequence is identified by its name followed by “/M” when the A40/A60/D61 amino acid combination is present in the corresponding heavy chain. Note: for EA5/M′, only positions A60/D61 are present.

FIG. 2 is the binding specificity of the antibodies of the invention as determined by surface plasmon resonance detection using a BIAcore 1000 instrument. The results for antibodies G5, 1E11, 4C10, 10D3, 12G3 and 4B11 and the corresponding substituted antibodies are shown in panel A while the results for EA5, MEDI-522 and their corresponding substituted antibodies are shown in panels B and C respectively.

### DETAILED DESCRIPTION OF THE INVENTION

The present invention is based on the discovery that the substitution of certain amino acid residues of the heavy chain of an antibody results in a dramatic increase in the producibility of said antibody in a eukaryotic host cell. In addition, the inventors have also found unexpectedly, that the amino acid substitutions of the invention did not negatively affect antigen binding and could actually enhance the antigen binding properties of the modified antibody. Thus, the invention includes antibodies displaying increased producibility wherein binding affinity is decreased although still at useful levels, unchanged, or increased.

Accordingly, the present invention relates to antibodies or antibody fragments with improved producibility and a method for improving the producibility of an antibody or antibody fragment by modifying the heavy chain. The antibodies or antibody fragments generated by the method of the invention will have antigen binding characteristics that are either improved, unchanged, or altered to an acceptable degree. Using methods described and contemplated herein, the present invention also provides antibodies or antibody fragment comprising said modified heavy chain having improved producibility and improved or unchanged antigen binding characteristics.

Without wishing to be bound by any particular theory, the amino acid substitutions of the invention improve the producibility of an antibody or antibody fragment by altering one or more of the factors which limit antibody production in cells including but not limited to, the level of gene expression, the stability of the messenger RNA, the stability of the translated antibody protein, protein folding, level of protein aggregation, and the toxicity of the antibody to the host cell.

It will be understood that the antibody residue numbers referred to herein are those of Kabat et. al. supra. In addition, the identity of certain individual residues at any given Kabat site number may vary from antibody chain to antibody chain due to interspecies or allelic divergence. However, for the sake of clarity and simplicity the residue numbers and identities of the Kabat human IgG heavy chain sequences will be used herein. Note that complementarity determining regions (CDRs) vary considerably from antibody to antibody (and by definition will not exhibit homology with the Kabat consensus sequences). Maximal alignment of framework residues frequently requires the insertion of “spacer” residues in the numbering system, to be used for the Fv region. It will be understood that the CDRs referred to herein are those of Kabat et al. supra.

In the case where there are two or more definitions of a term that are used and/or accepted within the art, the definition of the term as used herein is intended to include all such meanings unless explicitly stated to the contrary. A specific example is the use of the term “CDR” to describe the non-contiguous antigen combining sites found within the variable region of both heavy and light chain polypeptides. This particular region has been described by Kabat et al., 1991, NIH Publication 91-3242, National Technical Information Service, Springfield, Va.) and by Chothia et al. (1987, J. Mol. Biol. 196:901-17) and additionally by MacCallum et al.(1996, J. Mol. Biol. 262:732-45), each of which are incorporated herein by reference, where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues that encompass the CDRs as defined by each of the above cited references are set forth below in Table 1 as a comparison. The exact residue numbers which encompass a particular CDR will vary depending on the sequence and size of the CDR.

Those skilled in the art can routinely determine which residues comprise a particular CDR given the variable region amino acid sequence of the antibody.

TABLE 1
CDR Definitions

| | Kabat¹ | Chothia² | MacCallum³ |
|---|---|---|---|
| VH CDR1 | 31-35 | 26-32 | 30-35 |
| VH CDR2 | 50-65 | 53-55 | 47-58 |
| VH CDR3 | 95-102 | 96-101 | 93-101 |
| VL CDR1 | 24-34 | 26-32 | 30-36 |
| VL CDR2 | 50-56 | 50-52 | 46-55 |
| VL, CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

In one embodiment, antibodies of the invention will have at least one amino acid substitution wherein said substituted antibody has increased production levels compared to the antibody without said substitution.

In a specific embodiment, antibodies of the invention are substituted at one or more positions from the group consisting of: 40H, 60H, and 61H, utilizing the numbering system set forth in Kabat. More specifically, one or more of the amino acid residues 40H, 60H and 61H are substituted with alanine, alanine and aspartic acid, respectively.

In another embodiment, the invention provides a method for producing a substituted antibody with increased production levels.

In a preferred embodiment, the invention provides a method for increasing the producibility of an antibody or antibody fragment comprising the steps of: (a) substituting where necessary the amino acid residues at positions 40H, 60H, and 61 H, utilizing the numbering system set forth in Kabat, of the antibody of interest with alanine, alanine and aspartic acid, respectively; and (b) cultivating the host cell under conditions where the modified antibody polypeptide is expressed by said host cell.

It is specifically contemplated that one may choose to analyze the nature of the amino acids at positions 40H, 60H, and 61H prior to making any substitutions.

One skilled in the art would appreciate that in some cases the antibody of interest will already have the appropriate sequence at one or more of the aforementioned positions. In this situation, substitution(s) will only be introduced at the remaining non matching position(s) (e.g., at positions 40H/60H, 40H/61H, 60H/61H, 40H, 60H, or 61H).

In a preferred embodiment, the amino acid residue at positions 40H, 60H and 61H will be substituted with alanine, alanine and aspartic acid respectively.

In other preferred embodiments, the amino acid residues at position 40H and 60H will be substituted with alanine or 40H and 61H will be substituted with alanine and aspartic acid respectively or 60H and 61H will be substituted with alanine and aspartic acid respectively.

In still other preferred embodiments, the amino acid residues at position 40H or 60H will be substituted with alanine or 61H will be substituted with aspartic acid.

It is specifically contemplated that conservative amino acid substitutions may be made for said amino acid substitutions at positions 40H, 60H and/or 61H of the antibody of interest, described supra. It is well known in the art that “conservative amino acid substitution” refers to amino acid substitutions that substitute functionally-equivalent amino acids. Conservative amino acid changes result in silent changes in the amino acid sequence of the resulting peptide. For example, one or more amino acids of a similar polarity act as functional equivalents and result in a silent alteration within the amino acid sequence of the peptide. Substitutions that are charge neutral and which replace a residue with a smaller residue may also be considered “conservative substitutions” even if the residues are in different groups (e.g., replacement of phenylalanine with the smaller isoleucine). Families of amino acid residues having similar side chains have been defined in the art. Several families of conservative amino acid substitutions are shown in Table 2.

TABLE 2
Families of Conservative Amino Acid Substitutions

| Family | Amino Acids |
|---|---|
| non-polar | Trp, Phe, Met, Leu, Ile, Val, |
| | Ala, Pro |
| uncharged polar | Gly, Ser, Thr, Asn, Gln, Tyr, |
| | Cys |
| acidic/negatively charged | Asp, Glu |
| basic/positively charged | Arg, Lys, His |
| Beta-branched | Thr, Val, Ile |
| residues that influence chain orientation | Gly, Pro |
| aromatic | Trp, Tyr, Phe, His |

The term “conservative amino acid substitution” also refers to the use of amino acid analogs or variants. Guidance concerning how to make phenotypically silent amino acid substitutions is provided in Bowie et al. , “Deciphering the Message in Protein Sequences: Tolerance to Amino Acid Substitutions,” (1990, Science 247:1306-10).

In still another preferred embodiment, the method of the invention will result in an antibody with increased expression levels and/or purification yields.

In a more preferred embodiment, the method of the invention will result in an increase in antibody expression levels in crude media samples as determined by ELISA and/or purified antibody yields of at least 2 fold, or of at least 4 fold, or of at least 5 fold, or of at least 10 fold, or of at least 25 fold, or of at least 50 fold or of at least 100 fold when compared to the antibody without said substitution.

One skilled in the art will understand that amino acid substitutions and other modifications of an antibody may alter its antigen binding characteristics (examples of binding characteristics include but are not limited to, binding specificity, equilibrium dissociation constant (K_{D}), dissociation and association rates (K_{off} and Kₒₙ respectively), binding affinity and/or avidity) and that certain alterations are more or less desirable. For example a modification that preserves or enhances antigen binding would be more preferable then one that diminished or altered antigen binding. The binding characteristics of an antibody for a target antigen, may be determined by a variety of methods including but not limited it, equilibrium methods (e.g., enzyme-linked immunoabsorbent assay (ELISA) or radioimmunoassay (RIA)), or kinetics (e.g., BIACORE® analysis; see Example 2), for example. Other commonly used methods to examine the binding characteristics of antibodies are described in Using Antibodies: A Laboratory Manual, Cold Spring Harbor Laboratory Press, NY, Harrow et al., 1999 and Antibodies: A Laboratory Manual, Cold Spring Harbor Laboratory Press, NY; Harlow et al., 1989.

It is well known in the art that the equilibrium dissociation constant (K_{D}) is defined as k_{off}/kₒₙ. It is generally understood that an antibody with a low K_{D} is preferable to an antibody with a high K_{D}. However, in some instances the value of the kₒₙ or k_{off} may be more relevant than the value of the K_{D}. One skilled in the art can determine which kinetic parameter is most important for a given antibody and application. In a preferred embodiment, the method of the invention will result in antibodies with improved producibility and one or more antigen binding characteristics (e.g., binding specificity, K_{D}, K_{off}, Kₒₙ, binding affinity and/or avidity) that are improved by at least 2%, or by at least 5%, or by at least 10%, or by at least 20%, or by at least 30%, or by at least 40%, or by at least 50%, or by at least 60%, or by at least 70%, or by at least 80% when compared to kinetic parameters of the antibody without said substitution.

In another embodiment, the method of the invention will result in antibodies with at least one amino acid residue substitution that increase expression levels and/or purification yields, but do not substantially diminish the antigen binding of the antibody. For example, the method of the invention will generate antibodies that exhibit increase expression levels and/or purification yields, but preferably have no reduction in any antigen binding characteristic (e.g., binding specificity, K_{D}, K_{off}, Kₒₙ, binding affinity and/or avidity), or have one or more antigen binding characteristics that are reduced by less than 1%, or by less than 5%, or by less than 10%, or by less than 20%, or by less than 30%, or by less than 40%, or by less than 50%, or by less than 60%, or by less than 70%, or by less than 80% when compared to antigen binding of the antibody without said substitution.

The skilled artisan will further appreciate that the method of the invention may also be combined with other methods to increase the producibility of an antibody. Such methods include but are not limited to, manipulation of the growth media and/or conditions, modifications of the host cell, the introduction of additional amino acid substitutions or mutations into the heavy and/or light chains of the antibody and other modifications of the antibody. Additionally, the method of the invention may be combined with additional methods to generate an antibody with other preferred characteristics including but not limited to: increased serum half life, increase binding affinity, reduced immunogenicity, increased production, and altered binding specificity (for examples see infra).

The present invention also provides new antibody polypeptides having at least one amino acid residue substitution that results in improved producibility in host cells as compared to the antibody without said substitution.

The present invention further provides new antibody polypeptides having at least one amino acid residue substitution that results in improved producibility in host cells and improvements in one or more antigen binding characteristics (e.g., binding specificity, K_{D}, K_{off}, Kₒₙ, binding affinity and/or avidity) as compared to the antibody without said substitution.

In a preferred embodiment, the invention refers to antibody polypeptides having at least one amino acid residue substitution, characterized in that their expression levels in crude media samples as determined by ELISA and/or purified antibody yields exceed the expression levels and/or purification yields of the chimeric antibodies without substitutions by at least 100 fold, or by at least 50 fold, or by at least 25 fold, or by least 10 fold, or by at least 5 fold, or by at least 4 fold, or by at least 2 fold.

In a preferred embodiment, antibodies of the invention have both improved producibility and one or more antigen binding characteristics (e.g., binding specificity, K_{D}, K_{off}, Kₒₙ, binding affinity and/or avidity) that are improved by at least 2%, or by at least 5%, or by at least 10%, or by at least 20%, or by at least 30%, or by at least 40%, or by at least 50%, or by at least 60%, or by at least 70%, or by at least 80% when compared to kinetic parameters of the antibody without said substitutions.

In another embodiment, antibodies of the invention will exhibit increased expression levels and/or purification yields, but preferably have no reduction in any antigen binding characteristic (e.g., binding specificity, K_{D}, K_{off}, Kₒₙ, binding affinity and/or avidity), or have one or more antigen binding characteristics that are reduced by less than 1%, or by less than 5%, or by less than 10%, or by less than 20%, or by less than 30%, or by less than 40%, or by less than 50%, or by less than 60%, or by less than 70%, or by less than 80% when compared to antigen binding of the antibody without said substitution.

It is also specifically contemplated that the modified antibodies of the invention may contain inter alia additional amino acid residue substitutions, mutations and/or modifications which result in an antibody with preferred characteristics including but not limited to: increased serum half life, increase binding affinity, reduced immunogenicity, increased production, and binding specificity (for examples see infra).

In one embodiment, the modified antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter it's glycosylation, again to alter one or more functional properties of the antibody. Each of these embodiments is described in further detail below. The numbering of residues in the Fc region is that of the EU index of Kabat.

In one embodiment, the amino acid sequence of the Fc region is modified by deleting, adding and/or substituting at least amino acid residue to alter one or more of the functional properties of the antibody described above. This approach is described further in Duncan et al, 1988, Nature 332:563-564; Lund et al., 1991, J. Immunol 147:2657-2662; Lund et al, 1992, Mol Immunol 29:53-59; Alegre et al, 1994, Transplantation 57:1537-1543; Hutchins et al., 1995, Proc Natl. Acad Sci USA 92:11980-11984; Jefferis et al, 1995, Immunol Lett. 44:111-117; Lund et al., 1995, Faseb J 9:115-119; Jefferis et al, 1996, Immunol Lett 54:101-104; Lund et al, 1996, J Immunol 157:4963-4969; Armour et al., 1999, Eur J Immunol 29:2613-2624; Idusogie et al, 2000, J Immunol 164:4178-4184; Reddy et al, 2000, J Immunol 164:1925-1933; Xu et al., 2000, Cell Immunol 200:16-26; Idusogie et al, 2001, J Immunol 166:2571-2575; Shields et al., 2001, J Biol Chem 276:6591-6604; Jefferis et al, 2002, Immunol Lett 82:57-65; Presta et al., 2002, Biochem Soc Trans 30:487-490); U.S. Pat. Nos. 5,624,821; 5,885,573; 5,677,425; 6,165,745; 6,277,375; 5,869,046; 6,121,022; 5,624,821; 5,648,260; 6,194,551; 6,737,056 U.S. patent application Ser. Nos. 10/370,749 and PCT Publications WO 94/2935; WO 99/58572; WO 00/42072; WO 04/029207, each of which is incorporated herein by reference in its entirety.

In still another embodiment, the glycosylation of the modified antibodies of the invention is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861, each of which is incorporated herein by reference in its entirety.

Additionally or alternatively, a modified antibody of the invention can be made that has an altered type of glycosylation, such as a hypofucosylated antibody having reduced amounts of fucosyl residues or an antibody having increased bisecting GlcNAc structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent No: EP 1,176,195; PCT Publications WO 03/035835; WO 99/54342 80, each of which is incorporated herein by reference in its entirety.

Preferred Antibodies of the Invention

Antibodies modified by the method of the present invention and generated by the method of the invention may include, but are not limited to, synthetic antibodies, monoclonal antibodies, recombinantly produced antibodies, intrabodies, multispecific antibodies, bispecific antibodies, human antibodies, humanized antibodies, chimeric antibodies, synthetic antibodies, single-chain Fvs (scFv), Fab fragments, F(ab′) fragments, disulfide-linked Fvs (sdFv), and anti-idiotypic (anti-Id) antibodies, and epitope-binding fragments of any of the above. In particular, antibodies used in the methods of the present invention include immunoglobulin molecules and immunologically active portions of immunoglobulin molecules. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

Antibodies or antibody fragments modified by the method of the invention and generated by the method of the present invention may be from any animal origin including birds and mammals (e.g., human, murine, donkey, sheep, rabbit, goat, guinea pig, camel, horse, or chicken). Preferably, the antibodies are human or humanized monoclonal antibodies. As used herein, “human” antibodies include antibodies having the amino acid sequence of a human immunoglobulin and include antibodies isolated from human immunoglobulin libraries or from mice that express antibodies from human genes.

Antibodies or antibody fragments modified by the method of the invention and generated by the method of the present invention may be monospecific, bispecific, trispecific or of greater multispecificity. Multispecific antibodies may immunospecifically bind to different epitopes of desired target molecule or may immunospecifically bind to both the target molecule as well as a heterologous epitope, such as a heterologous polypeptide or solid support material. See, e.g., International Publication Nos. WO 93/17715, WO 92/08802, WO 91/00360, and WO 92/05793; Tutt, et al., 1991, J. Immunol. 147:60-69; U.S. Pat. Nos. 4,474,893, 4,714,681, 4,925,648, 5,573,920, and 5,601,819; and Kostelny et al., 1992, J. Immunol. 148:1547-1553.

The method and antibodies of the present invention encompasses single domain antibodies, including camelized single domain antibodies (see e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079; which are incorporated herein by reference in their entireties).

The method and antibodies of the present invention also encompass the use of antibodies or fragments thereof that have half-lives (e.g., serum half-lives) in a mammal, preferably a human, of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies of the present invention or fragments thereof in a mammal, preferably a human, results in a higher serum titer of said antibodies or antibody fragments in the mammal, and thus, reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor (see, e.g., International Publication No. WO 97/34631 and U.S. patent application Ser. No. 10/020,354, both of which are incorporated herein by reference in their entireties).

The method and antibodies of the present invention also encompasses antibodies that are bispecific comprising a modified antibody of the invention, or antigen-binding portion thereof, linked to a second functional moiety having a different binding specificity than said antibody, or antigen binding portion thereof, of the invention. In a further embodiment, the invention encompasses antibodies which are multispecific, where the antibody molecule further comprises a third, or a fourth, or more function moiety having a different binding specificity than said antibody of the invention, or antigen binding portion thereof.

In a specific embodiment, method and antibodies of the present invention are bispecific T cell engagers (BiTEs). Bispecific T cell engagers (BiTE) are bispecific antibodies that can redirect T cells for antigen-specific elimination of targets. A BiTE molecule has an antigen-binding domain that binds to a T cell antigen (e.g. CD3) at one end of the molecule and an antigen binding domain that will bind to an antigen on the target cell. A BiTE molecule was recently described in WO 99/54440, which is herein incorporated by reference. This publication describes a novel single-chain multifunctional polypeptide that comprises binding sites for the CD19 and CD3 antigens (CD19×CD3). This molecule was derived from two antibodies, one that binds to CD19 on the B cell and an antibody that binds to CD3 on the T cells. The variable regions of these different antibodies are linked by a polypeptide sequence, thus creating a single molecule. Also described, is the linking of the variable heavy chain (VH) and light chain (VL) of a specific binding domain with a flexible linker to create a single chain, bispecific antibody.

In another embodiment, the BiTE molecule can comprise a molecule that binds to other T cell antigens (other than CD3). For example, ligands and/or antibodies that immunospecifically bind to T-cell antigens like CD2, CD4, CD8, CD11a, TCR, and CD28 are contemplated to be part of this invention. This list is not meant to be exhaustive but only to illustrate that other molecules that can immunospecifically bind to a T cell antigen can be used as part of a BiTE molecule. These molecules can include the VH and/or VL portions of the antibody or natural ligands (for example LFA3 whose natural ligand is CD3).

Methods of Generating Antibodies

Antibodies or antibody fragments modified by the method of the invention and generated by the invention can be generated by any method known in the art for the synthesis of antibodies, in particular, by chemical synthesis or preferably, by recombinant expression techniques.

Monoclonal antibodies modified by the method of the present invention can be. prepared using a wide variety of techniques known in the art including the use of hybridoma, recombinant, and phage display technologies, or a combination thereof. For example, monoclonal antibodies can be produced using hybridoma techniques including those known in the art and taught, for example, in Antibodies: A Laboratory Manual, E. Harlow and D. Lane, ed., Cold Spring Harbor Laboratory Press (Cold Spring Harbor, N.Y., 1988); and Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a antigen of interest, generally but not always a polypeptide such as a full length protein or a domain thereof (e.g., the extracellular domain) can be utilized, and once an immune response is detected, e.g., antibodies specific for the antigen of interest are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. Additionally, a RIMMS (repetitive immunization, multiple sites) technique can be used to immunize an animal (Kilpatrick et al., 1997, Hybridoma 16:381-9, incorporated herein by reference in its entirety). Hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, monoclonal antibodies can be generated by culturing a hybridoma cell secreting an antibody of interest wherein, preferably, the hybridoma is generated by fusing splenocytes isolated from a mouse immunized with polypeptide of interest or fragment thereof with myeloma cells and then screening the hybridomas resulting from the fusion for hybridoma clones that secrete an antibody able to bind the polypeptide of interest.

Antibody fragments of the invention may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles that carry the polynucleotide sequences encoding them. In particular, DNA sequences encoding V_{H} and V_{L} domains are amplified from animal cDNA libraries (e.g., human or murine cDNA libraries of lymphoid tissues). The DNA encoding the V_{H} and V_{L} domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the V_{H} and V_{L} domains are usually recombinantly fused to either the phage gene III or gene VIII. Phage expressing an antigen binding domain that binds to the antigen epitope of interest can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177; Kettleborough et al., 1994, Eur. J Immunol. 24:952-958; Persic et al., 1997, Gene 187:9; Burton et al., 1994, Advances in Immunology 57:191-280; International Application No. PCT/GB91/01134; International Publication Nos. WO 90/02809, WO 91/10737, WO 92/01047, WO 92/18619, WO 93/11236, WO 95/15982, WO 95/20401, and WO 97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

In a preferred embodiment, after phage selection, the antibody coding regions from the phage are isolated and used to generate whole antibodies, including human antibodies as described in the above references. In another preferred embodiment the reconstituted antibody of the invention is expressed in any desired host, including bacteria, insect cells, plant cells, yeast, and in particular, mammalian cells (e.g., as described below). Techniques to recombinantly produce Fab, Fab′ and F(ab′)2 fragments can also be employed using methods known in the art such as those disclosed in International Publication No. WO 92/22324; Mullinax et al., 1992, BioTechniques 12:864; Sawai et al., 1995, AJRI 34:26; and Better et al., 1988, Science 240:1041 (said references incorporated by reference in their entireties).

The nucleotide sequence encoding an antibody of the invention can be obtained from sequencing hybridoma clone DNA. If a clone containing a nucleic acid encoding a particular antibody or an epitope-binding fragment thereof is not available, but the sequence of the antibody molecule or epitope-binding fragment thereof is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from any tissue or cells expressing the antibody, such as hybridoma cells selected to express an antibody) by PCR amplification using synthetic primers that hybridize to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g. recombinant DNA techniques, site directed mutagenesis, PCR, etc. (see, Or example, the techniques described in Current Protocols in Molecular Biology, F. M. Ausubel et al., ed., John Wiley & Sons (Chichester, England, 1998); Molecular Cloning: A Laboratory Manual, 3nd Edition, J. Sambrook et al., ed., Cold Spring Harbor Laboratory Press (Cold Spring Harbor, N.Y., 2001); Antibodies: A Laboratory Manual, E. Harlow and D. Lane, ed., Cold Spring Harbor Laboratory Press (Cold Spring Harbor, N.Y., 1988); and Using Antibodies: A Laboratory Manual, E. Harlow and D. Lane, ed., Cold Spring Harbor Laboratory (Cold Spring Harbor, N.Y., 1999) which are incorporated by reference herein in their entireties), to generate antibodies having a different amino acid sequence by, for example, introducing deletions, and/or insertions into desired regions of the antibodies.

In a preferred embodiment, antibodies of the invention include amino acid substitutions into the variable region of the heavy chain such that positions 41H, 60H and 61 H substituted by alanine, alanine and aspartic acid, respectively.

In a more preferred embodiment, the VH and VL nucleotide sequences are cloned and used to generate whole antibodies. Utilizing cloning techniques known to those skilled in the art, the PCR primers including V_{H} or V_{L} nucleotide sequences, a restriction site, and a flanking sequence to protect the restriction site are used to amplify the V_{H} or V_{L} sequences in scFv. The PCR amplified V_{H} domains are cloned into vectors expressing a V_{H} constant region, e.g., the human gamma 4 constant region, and the PCR amplified V_{L} domains are cloned into vectors expressing a V_{L} constant region, e.g., human kappa or lambda constant regions. The V_{H} and V_{L} domains may also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

It is specifically contemplated that for some uses, including in vivo use of antibodies in humans and in vitro detection assays, antibodies of the invention are preferably human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the J_{H} region prevents endogenous antibody production. The modified embryonic stem cells are expanded and microinjected into blastocysts to produce chimeric mice. The chimeric mice are then be bred to produce homozygous offspring that express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.), Genpharm (San Jose, Calif.) and Medarex (Princeton, N.J.) can be engaged to provide human antibodies directed against a selected antigen using technology similar to that described above.

A chimeric antibody is a molecule in which different portions of the antibody are derived from different immunoglobulin molecules such as, for example, antibodies having a variable region derived from a non-human antibody and a human immunoglobulin constant region. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, and 4,816,397, CDR-grafting (EP 239,400; International Publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28(4/5): 489-498; Studnicka et al., 1994, Protein Engineering 7:805; and Roguska et al., 1994, PNAS 91:969), and chain shuffling (U.S. Pat. No. 5,565,332). which are incorporated herein by reference in their entirety.

Methods of Expressing Antibodies

Recombinant expression of an antibody requires construction of an expression vector containing a nucleotide sequence that encodes the antibody. Once a nucleotide sequence encoding an antibody molecule or a heavy or light chain of an antibody, or portion thereof (preferably containing the heavy or light chain variable regions) has been obtained, the vector for the production of the antibody molecule may be produced by recombinant DNA technology using techniques well known in the art. Thus, methods for preparing a protein by expression a polynucleotide containing an antibody encoding nucleotide sequence are described herein. Methods, which are well known to those skilled in the art, can be used to construct expression vectors containing antibody coding sequences and appropriate transcriptional and translational control signals. These methods include, for example, in vitro recombinant DNA techniques, synthetic techniques, and in vivo genetic recombination.

The invention, thus, provides replicable vectors comprising a nucleotide sequence encoding a modified antibody molecule with one or more modifications in the amino acid residues 40H, 60H and 61H of the heavy chain. The nucleotide sequence encoding the heavy-chain variable region, light-chain variable region, both the heavy-chain and light-chain variable regions, an epitope-binding fragment of the heavy- and/or light-chain variable region, or one or more complementarily determining regions (CDRs) of an antibody may be cloned into such a vector for expression.

The antibody expression vector is transferred to a host cell by conventional techniques and the transfected cells are then cultured by conventional techniques to produce a substituted antibody have improved producibility. A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleotide coding sequences express an antibody molecule of the invention in situ.

In a preferred embodiment, antibodies generated by the method of the invention are expressed in eukaryotic host cells. In a more preferred embodiment the host cell is mammalian. Mammalian cell systems include but are not limited to, CHO, BHK, HeLa, COS, MDCK, NIH 3T3, W138, NS0, SP/20 and other lymphocytic cells, and human cells such as PERC6, HEK 293 harboring recombinant expression constructs containing promoters derived from the genome of mammalian cells (e.g., metallothionein promoter) or from mammalian viruses (e.g., the adenovirus late promoter; the vaccinia virus 7.5K promoter). For example, mammalian cells such as Chinese hamster ovary cells (CHO) in conjunction with a vector such as the major intermediate early gene promoter element from human cytomegalovirus is an effective expression system for antibodies (Foecking et al., 1986, Gene, 45:101; and Cockett et al., 1990, BioTechnology, 8:2).

In mammalian host cells, a number of viral-based expression systems may be utilized to express an antibody molecule of the invention. In cases where an adenovirus is used as an expression vector, the antibody coding sequence of interest may be ligated to an adenovirus transcription/translation control complex, (e.g., the late promoter and tripartite leader sequence). This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g. see Logan & Shenk, 1984, Proc. Natl. Acad. Sci. USA, 1:355-59). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bitter et al., 1987, Methods in Enzymol., 153:516-44).

In addition, a host cell strain may be chosen which modulates the expression of the antibody sequences, or modifies and processes the antibody in the specific fashion desired. Such modifications (e.g., glycosylation) and processing (e.g., cleavage) of protein products may be important for the function of the antibody. Different host cells have characteristic and specific mechanisms for the post-translational processing and modification of proteins and gene products. Appropriate cell lines or host systems can be chosen to ensure the correct modification and processing of the antibody expressed. To this end, it is specifically contemplated that eukaryotic host cells which possess the cellular machinery for proper processing of the primary transcript, glycosylation, and phosphorylation of the gene product are be used. Such mammalian host cells include but are not limited to, CHO, BHK, HeLa, COS, MDCK, NIH 3T3, W138, NSO, SP/20 and other lymphocytic cells, and human cells such as PERC6, HEK 293.

For long-term, high-yield production of recombinant antibodies, stable expression is preferred. For example, cell lines that stably express the antibody molecule may be engineered. Rather than using expression vectors which contain viral origins of replication, host cells can be transformed with DNA controlled by appropriate expression control elements (e.g., promoter, enhancer, sequences, transcription terminators, −85 polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., 1977, Cell, 11:223), hypoxanthine guanine phosphoribosyltransferase (Szybalska & Szybalski, 1992, Proc. Natl. Acad. Sci. USA, 48:202), and adenine phosphoribosyltransferase (Lowy et al., 1980, Cell, 22:8-17) genes can be employed in tk-, hgprt- or aprt- cells, respectively. Also, anti-metabolite resistance can be used as the basis of selection for the following genes: dhfr, which confers resistance to methotrexate (Wigler et al., 1908, Proc. Natl. Acad. Sci. USA, 77:357 and O'Hare et al., 1981, Proc. Natl. Acad. Sci. USA, 78:1527), gpt, which confers resistance to mycophenolic acid (Mulligan & Berg, 1981. Proc. Natl. A cad. Sci. USA, 78:2072); neo, which confers resistance to the aminoglycoside G-418 (Wu and Wu, 1991, Biotherapy, 3:87-95; Tolstoshev, 1993, Ann. Rev. Pharmacol. Toxicol., 32:573-96; Mulligan, 1993, Science, 260:926-32; and Morgan and Anderson, 1993, Ann. Rev. Biochem., 62: 191-217; and May, 1993, TIB TECH, 11(5):155-2); and hygro, which confers resistance to hygromycin (Santerre et al., 1984, Gene, 30:147). Methods commonly known in the art of recombinant DNA technology may be routinely applied to select the desired recombinant clone, and such methods are described, for example, in Ausubel, F. M., et al., 1998, Current Protocols in Molecular Biology, John Wiley & Sons, and Sambrook, et al., 2001, Molecular Cloning: A Laboratory Manual, 3nd Edition, which are incorporated by reference herein in their entireties.

The expression levels of an antibody molecule can be further increased by vector amplification (for a review, see Bebbington and Hentschel, 1987, The use of vectors based on gene amplification for the expression of cloned genes in mammalian cells in DNA cloning, Vol. 3. Academic Press, New York). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol., 3:257)

The host cell may be co-transfected with two expression vectors of the invention, the first vector encoding a heavy chain derived polypeptide and the second vector encoding a light chain derived polypeptide. The two vectors may contain identical selectable markers, which enable equal expression of heavy and light chain polypeptides.

Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature, 322:52; and Kohler, 1980, Proc. Natl. Acad. Sci. USA, 77:2 197). The coding sequences for the heavy and light chains may comprise cDNA or genomic DNA.

In one embodiment, the whole recombinant antibody molecule, is expressed. In another embodiment, fragments (e.g., Fab fragments, F(ab′) fragments, and epitope-binding fragments) of the immunoglobulin molecule are expressed.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A purification, and sizing column chromatography), centrifugation, differential solubility, or by any other standard techniques for the purification of proteins. Further, the antibodies of the present invention or fragments thereof may be fused to heterologous polypeptide sequences described herein or otherwise known in the art to facilitate purification.

Antibody Derivatives

Antibodies modified by the method of the present invention and generated by the method of the invention include derivatives that are modified (i.e., by the covalent attachment of any type of molecule to the antibody such that covalent attachment). For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

Antibodies or fragments thereof with increased in vivo half-lives can be generated by attaching to said antibodies or antibody fragments polymer molecules such as high molecular weight polyethylene glycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography.

The present invention encompasses antibodies modified by the method of the present invention and generated by the method of the invention (or fragments thereof) recombinantly fused or chemically conjugated (including both covalent and non-covalent conjugations) to a heterologous polypeptide (or portion thereof, preferably to a polypeptide of at least 10, at least 20, at least 30, at least 40, at least 50, at least 60, at least 70, at least 80, at least 90 or at least 100 amino acids) to generate fusion proteins. The fusion does not necessarily need to be direct, but may occur through linker sequences. For example, antibodies may be used to target heterologous polypeptides to particular cell types, either in vitro or in vivo, by fusing or conjugating the antibodies to antibodies specific for particular cell surface receptors. Antibodies fused or conjugated to heterologous polypeptides may also be used in in vitro immunoassays and purification methods using methods known in the art. See e.g., International Publication WO 93/21232; EP 439,095; Naramura et al., 1994, Immunol. Lett. 39:91-99; U.S. Pat. No. 5,474,981; Gillies et al., 1992, PNAS 89:1428-1432; and Fell et al., 1991, J. Immunol. 146:2446-2452, which are incorporated by reference in their entireties.

The present invention further includes compositions comprising heterologous polypeptides fused or conjugated to antibody fragments. For example, the heterologous polypeptides may be fused or conjugated to a Fab fragment, Fd fragment, Fv fragment, F(ab)₂ fragment, or portion thereof. Methods for fusing or conjugating polypeptides to antibody portions are known in the art. See, e.g., U.S. Pat. Nos. 5,336,603, 5,622,929, 5,359,046, 5,349,053, 5,447,851, and 5,112,946; EP 307,434; EP 367,166; International Publication Nos. WO 96/04388 and WO 91/06570; Ashkenazi et al., 1991, PNAS 88: 10535-10539; Zheng et al., 1995, J. Immunol. 154:5590-5600; and Vil et al., 1992, PNAS 89:11337-11341 (said references incorporated by reference in their entireties).

DNA shuffling may be employed to alter the activities of antibodies of the invention or fragments thereof (e.g., antibodies or fragments thereof with higher affinities and lower dissociation rates). See, generally, U.S. Pat. Nos. 5,605,793; 5,811,238; 5,830,721; 5,834,252; and 5,837,458, and Patten et al., 1997, Curr. Opinion Biotechnol. 8:724-33; Harayama, 1998, Trends Biotechnol. 16:76; Hansson, et al., 1999, J. Mol. Biol. 287:265; and Lorenzo and Blasco, 1998, BioTechniques 24:308 (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination.

Moreover, the antibodies or fragments thereof can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, PNAS 86:821, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

In other embodiments, antibodies modified by the method of the present invention and generated by the method of the invention or fragments or variants thereof can be conjugated to a diagnostic or detectable agent. Such antibodies can be useful for monitoring or prognosing the development or progression of a cancer as part of a clinical testing procedure, such as determining the efficacy of a particular therapy. Such diagnosis and detection can accomplished by coupling the antibody to detectable substances including, but not limited to various enzymes, such as but not limited to horseradish peroxidase, alkaline phosphatase, beta-galactosidase, or acetylcholinesterase; prosthetic groups, such as but not limited to streptavidin/biotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I), lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (169Yb, ¹⁷⁵Yb), yttrium (⁹⁰Y), zinc (⁶⁵Zn); positron emitting metals using various positron emission tomographies, and nonradioactive paramagnetic metal ions.

The present invention further encompasses uses of modified antibodies of the invention or fragments thereof conjugated to a therapeutic agent.

An antibody or fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Examples include paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Further, an antibody or fragment thereof may be conjugated to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, for example, a toxin such as abrin, ricin A, Onconase (or another cytoxic RNase), pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Immunol., 6:1567), and VEGI (see, International Publication No. WO 99/23105), a thrombotic agent or an anti-angiogenic agent, e.g., angiostatin or endostatin; or, a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”)), or a growth factor (e.g., growth hormone (“GH”)).

Moreover, an antibody can be conjugated to therapeutic moieties such as a radioactive materials or macrocyclic chelators useful for conjugating radiometal ions (see above for examples of radioactive materials). In certain embodiments, the macrocyclic chelator is 1,4,7,10-tetraazacyclododecane-N,N′,N″,N″-tetraacetic acid (DOTA) which can be attached to the antibody via a linker molecule. Such linker molecules are commonly known in the art and described in Denardo et al., 1998, Clin Cancer Res. 4:2483-90; Peterson et al., 1999, Bioconjug. Chem. 10:553; and Zimmerman et al., 1999, Nucl. Med. Biol. 26:943-50 each incorporated by reference in their entireties.

Techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Alternatively, an antibody can be conjugated to a second antibody to form an antibody heteroconjugate as described by Segal in U.S. Pat. No. 4,676,980, which is incorporated herein by reference in its entirety.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### EXAMPLES

The invention is now described with reference to the following examples. These examples are provided for the purpose of illustration only and the invention should in no way be construed as being limited to these examples but rather should be construed to encompass any and all variations which become evident as a result of the teachings provided herein.

### Example 1

### Generation and Expression of the Various Antibody Constructs

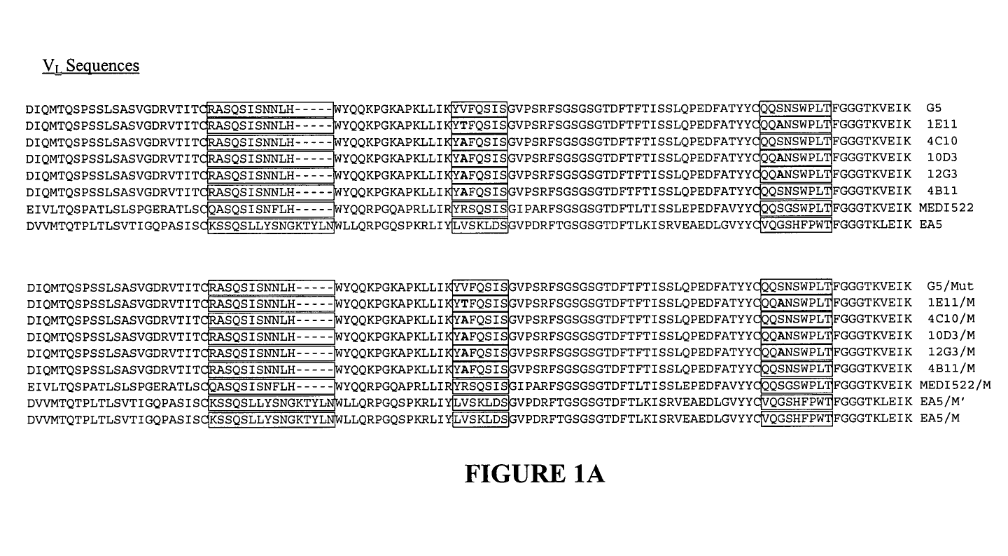

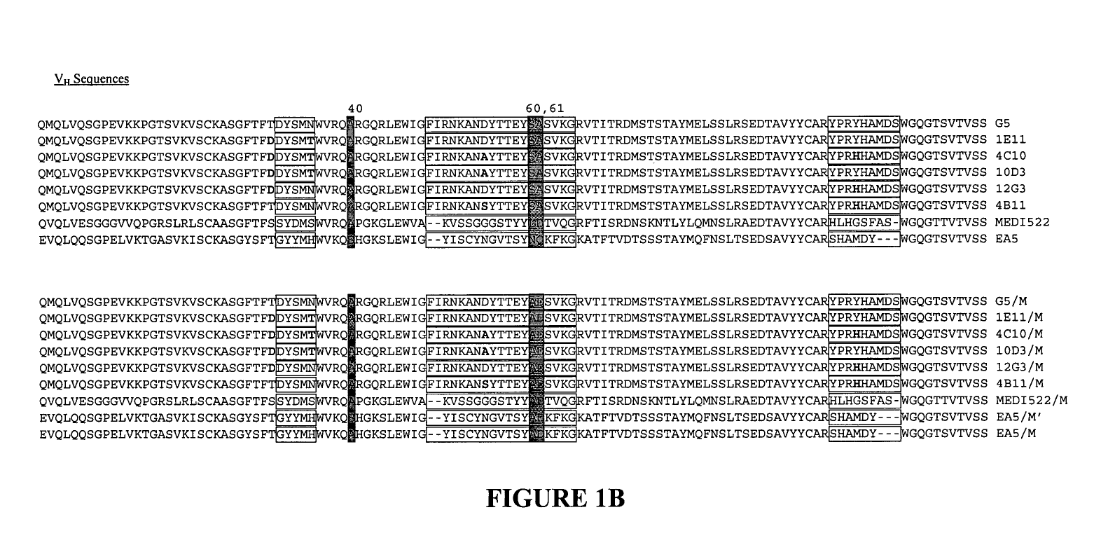

Six humanized monoclonal antibodies (G5, 10D3, 12G3, 1E11, 4C10, 4B11) and one human/mouse chimeric antibody (EA5) were generated against a common antigen, EphA2. All of these antibodies were poorly expressed in mammalian cells (see Table 3). Another humanized antibody, MEDI-522, which is expressed well in mammalian cells (see Table 3) was also used in these studies. One or more heavy chain substitutions at positions 40, 60 and/or 61 were generated in each of these antibodies to determine the effect on producibility by the presence of one or more preferred amino acid residues at these positions. Six of the humanized antibodies contained an Alanine at position H40, these antibodies were substituted with Alanine and Aspartate at positions H60 and H61 respectively. The last humanized antibody, MEDI-522 had both the H40 and H61 preferred amino acids, here position H60 was substituted with Alanine. The chimeric antibody, EA5, against the same antigen did not contain any of the preferred amino acids at positions H40, H60 or H61. Two separate heavy chains were generated for EA5, one which contained substitutions at positions 60 and 61 and another which contained substitutions at positions H40, H60 and H61. The specific amino acid residues of the heavy chain that were modified (see FIG. 1B) are described below. In all cases substitutions resulting in one or more preferred heavy chain residues at positions 40, 60 and 61 resulted in improved producibility (see Table 3). Interestingly, in the case of EA5 which contained none of the preferred amino acids, the heavy chain A60/D61 combination (EA5/M′ SEQ ID NO.: 31) by itself significantly increased production yields.

Materials and Methods

Generation, Characterization and Cloning of Antigen Specific Antibodies:

General methods for generating, screening, cloning and expressing antibodies are known to practitioners of the art. See, e.g., Current Protocols in Molecular Biology, F. M. Ausubel et al., ed., John Wiley & Sons (Chichester, England, 1998); Molecular Cloning: A Laboratory Manual, 3nd Edition, J. Sambrook et al., ed., Cold Spring Harbor Laboratory Press (Cold Spring Harbor, N.Y., 2001); Antibodies: A Laboratory Manual, E. Harlow and D. Lane, ed., Cold Spring Harbor Laboratory Press (Cold Spring Harbor, N.Y., 1988); and Using Antibodies: A Laboratory Manual, E. Harlow and D. Lane, ed., Cold Spring Harbor Laboratory (Cold Spring Harbor, N.Y., 1999) which are incorporated by reference herein in their entireties.

Generation of Heavychain Substitutions:

The variable regions of the light chains of antibody clones G5, 10D3, 12G3, 1E11, 4C10, 4B11, MEDI522 and EA5 (SEQ ID NOS. 1-8, respectively) and the variable regions of the heavy chains of antibody clones G5, 10D3, 12G3, 1E11, 4C10, 4B11, MEDI522 and EA5 (SEQ ID NOS. 9-16, respectively) were individually cloned into mammalian expression vectors encoding a human cytomegalovirus major immediate early (hCMVie) enhancer, promoter and 5′-untranslated region (Boshart et al., 1985, Cell 41:521-30). In this system, a human γ1 chain is secreted along with a human κ chain (Johnson et al., 1997, J. Infect. Dis. 176:1215-24). All of the heavy chain substitutions were introduced by site-directed mutagenesis using a Quick Change Multi Mutagenesis Kit (Stratagene, Calif.) according to the manufacturer's instructions. Specifically, S60A/A61D were introduced into clones G5, 10D3, 12G3, 1E11, 4C10 and 4B11 using the primer: 5′-ACACAACAGAGTACGCTGACTCTGTGAAGGGTAGAG TCACCATT-3′ (SEQ ID NO. 17); this generated heavy chain antibody clones G5/M, 10D3/M, 12G3/M, 1E11/M, 4C10/M and 4B11/M (SEQ ID NOS. 24-29); L60A was introduced into MEDI522 using the primers: 5′-GGTGGTGGTAGCACCTACTATGCA GACACTGTGCAGGGCCGATTCACC-3′ (SEQ ID NO.: 18) and 5′-GGTGAATCGG CCCTGCACAGTGTCTGCATAGTAGGTGCTACCACCACC-3′ (SEQ ID NO.: 19) generating MEDI522/M (SEQ ID NO. 30); N60A/Q61D were introduced into EA5 using the primers: 5′-GTTACAATGGTGTTACTAGCTACGCCGACAAGTTCAAGGGCAAGG CCAC-3′ (SEQ ID NO.: 20) and 5′-GTGGCCTTGCCCTTGAACTTGTCGGCGTAGCT AGTAACACCATTGTAAC-3′ (SEQ ID NO.: 21) generating EA5/M′ (SEQ ID NO.: 31); and S40A/N60A/Q61D were introduced into EA5 using the primers: 5′-CTACATGC ACTGGGTCAAGCAGGCCCATGGAAAGAGCCTTGAG-3′ (SEQ ID NO.: 22), 5′-CTCAAGGCTCTTTCCATGGGCCTGCTTGACCCAGTGCATGTAG-3′ (SEQ ID NO.: 23), 5′-GTTACAATGGTGTTACTAGCTACGCCGACAAGTTCAAGGGCAAGGCCAC-3′ (SEQ ID NO.:20) and 5′-GTGGCCTTGCCCTTGAACTTGTCGGCGTAGCTAGT AACACCATTGTAAC-3′ (SEQ ID NO.: 21) generating EA5/M (SEQ ID NO.: 32). Note that the light chains remain unaltered and are still represented by SEQ ID NOS 1-8) (FIG. 1A). The sequences were verified using an ABI 3100 sequencer. Human embryonic kidney (HEK) 293 cells were then transiently transfected with the various antibody constructs in 35 mm, 6-wells dishes using Lipofectamine and standard protocols. Supernatants were harvested twice at 72 and 144 hours post-transfection (referred to as 1^{st} and 2^{nd} harvest, respectively). The secreted, soluble human IgG1s were then assayed in terms of production yields and binding to original antigen (see below).

Measurement of the Expression Yields:

The expression yields of antibody clones G5, G5/M, 10D3, 10D3/M, 12G3, 12G3/M, 1E11, 1E11/M, 4C10, 4C10/M, 4B11 and 4B11/Mut were measured by ELISA. Transfection supernatants collected twice at three days intervals (see above) were assayed for antibody production using an anti-human IgG ELISA. Briefly, individual wells of a 96-well Biocoat plate (BD Biosciences, San Jose, Calif.) coated with a goat anti-human IgG were incubated with samples (supernatants) or standards (human IgG, 0.5-100 ng/ml), then with a horseradish peroxydase conjugate of a goat anti-human IgG antibody. Peroxydase activity was detected with 3,3′,5,5′-tetramethylbenzidine and the reaction was quenched with 0.2 M H₂SO₄. Plates were read at 450 nm. The results are summarized in Table 3.

TABLE 3
Producibility Improvements of Heavy Chain Modified Antibodies^{a}

| | Transfection | Transfection | Transfection | Transfection | Transfection | Fold |
|---|---|---|---|---|---|---|
| | #1 | #2 | #3 | #4 | #5 | increase^{d} |
| Modified | H1^{b} H2^{c} | H1 H2 | H1 H2 | H1 H2 | H1 H2 | H1 H2 |
| Antibody | μg/ml | μg/ml | μg/ml | μg/ml | μg/ml | μg/ml |
| G5 | 0.3-1.2 | 0.5-1.3 | 0.6-1.4 | | | |
| G5/M | 1.6-3.8 | 2.5-6.2 | | | | 4.4-3.8 |
| 1E11 | 0.7-2.0 | 1.2-3.4 | | | | |
| 1E11/M | 1.7-3.3 | 1.3-3.9 | | | | 1.6-1.3 |
| 4C10 | 2.0-3.0 | 2.4-3.2 | 2.1-3.3 | | | |
| 4C10/M | 3.2-5.8 | 3.8-7.3 | 5.0-4.6 | 6.8-7.8 | 5.1-7.7 | 2.2-2.1 |
| 10D3 | 0.7-1.7 | 1.4-3.5 | | | | |
| 10D3/M | 1.2-2.9 | 2.8-5.1 | | | | 2.0-1.5 |
| 12G3 | 0.9-2.3 | 1.8-3.6 | 1.4-2.4 | | | |
| 12G3/M | N.D. | 3.5-8.7 | 3.2-5.4 | 3.3-5.9 | 4.4-8.4 | 2.6-2.6 |
| 4B11 | 0.4-1.5 | 0.7-3.0 | | | | |
| 4B11/M | 1.0-2.3 | 2.4-5.2 | | | | 3.0-1.7 |
| MEDI522 | 14.8-12.2 | 10.9-8.8 | 16.9-11.8 | | | |
| MEDI522/M | 19.3-19.4 | 18.6-12.3 | 23.7-16.2 | | | 1.4-1.4 |
| EA5 | 2.7-2.8 | 1.0-1.2 | 4.0-2.9 | | | |
| EA5/M′ | 3.3-3.9 | 1.1-1.9 | 3.6-5.5 | | | 1.1-1.6 |
| EA5/M | 4.6-2.4 | 2.4-2.2 | 4.8-3.9 | | | 1.5-1.2 |

^{a}HEK 293 cells were transiently transfected with the various antibody constructs.
^{b}H1 = First Harvest (72 hours post-transfection).
^{c}H2 = Second Harvest (144 hours post-transfection).
^{d}Fold increase = average yield for each harvest (H1, H2) of the heavy chain modified “Mut” antibody divided by the average yield for each harvest of the unmodified antibody.

### Example 2

### Analysis of the Binding Characteristics of the Modified Antibodies

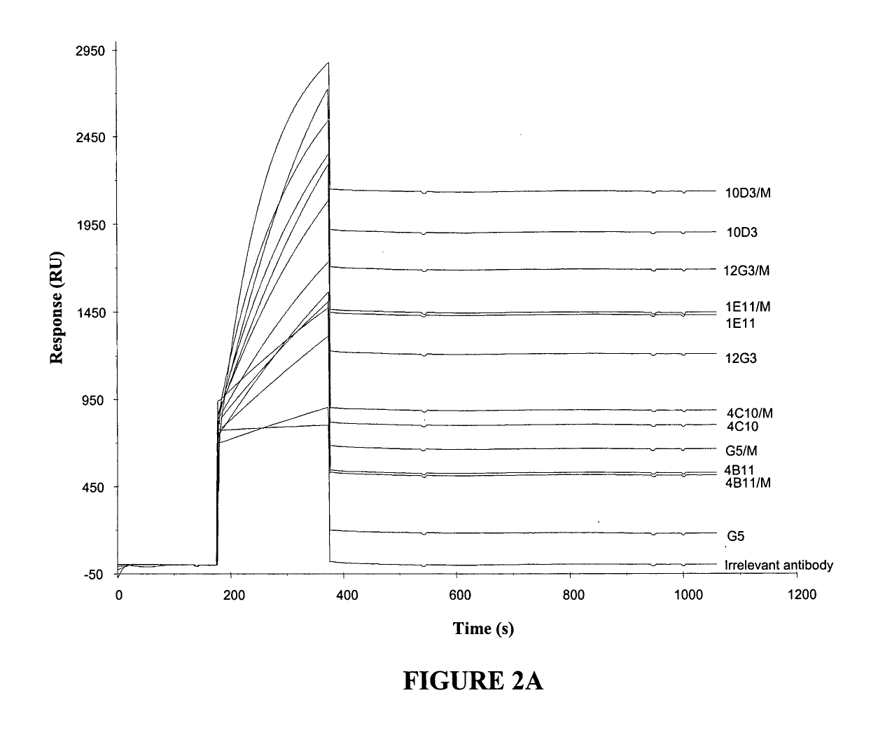

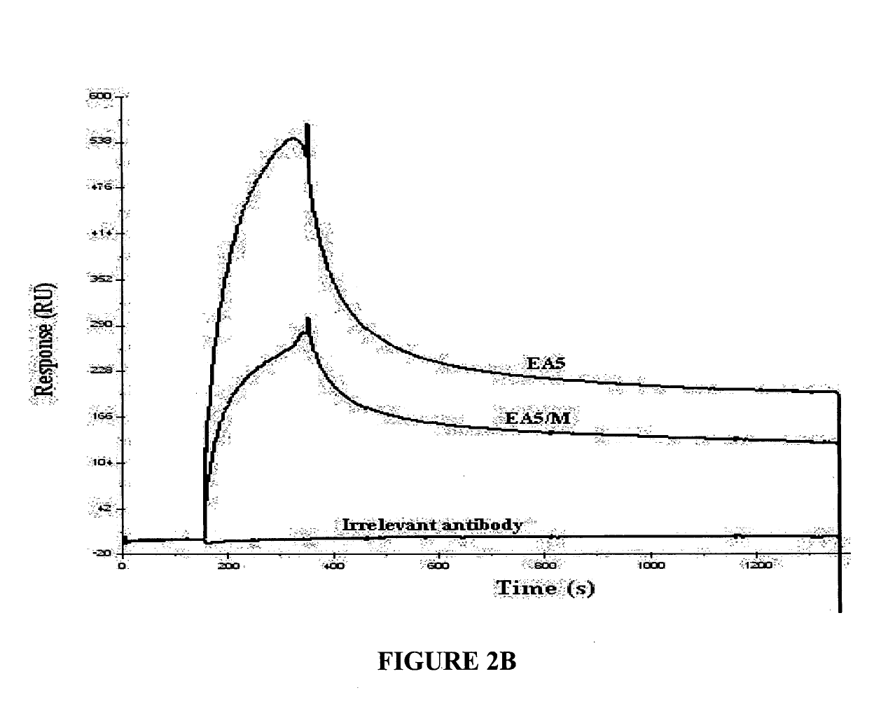

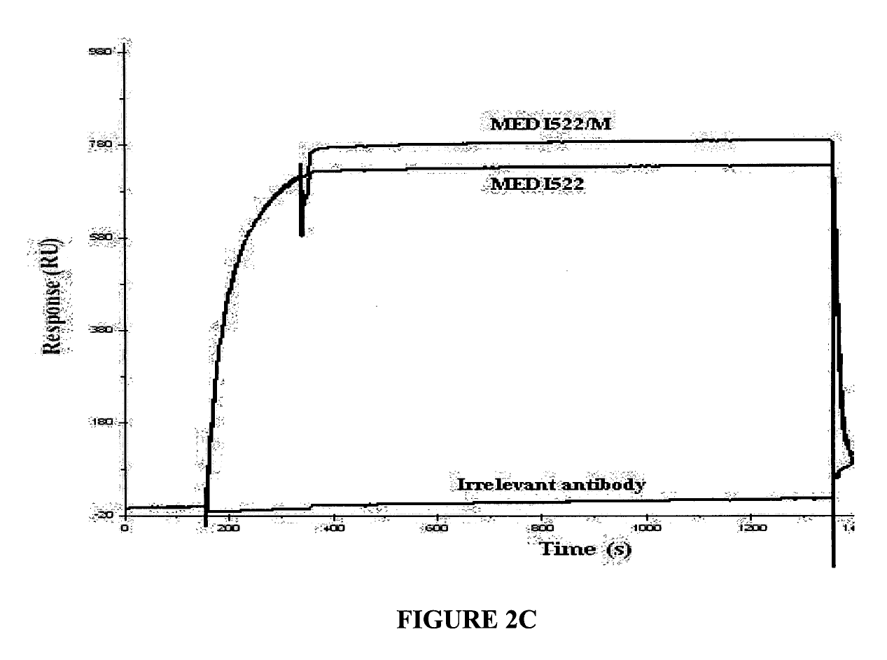

Because two of the heavy chain substitutions (positions 60H and 61H, Kabat numbering) fall within the CDRs as defined by Kabat, it was possible that the general binding characteristics of the substituted antibodies had been altered. Remarkably, the modifications improved the yields for each of the six antibodies without significantly altering the binding specificity (see FIGS. 2A-C). Two of the modified antibodies were chosen for more extensive analysis. Surprisingly, the binding constants of one were improved by at least 20%, while the other remained virtually unchanged (see Table 4).

Materials and Methods

Binding Specificity via BIAcore Analysis:

The interaction of immobilized EphA2-Fc or αvβ3 with IgG-containing transfection supernatants corresponding to clones G5, G5/M, 10D3, 10D3/M, 12G3, 12G3/M, 1E11, 1E11/M, 4C10, 4C10/M, 4B11 and 4B11/M (FIG. 2A), EA5/EA5M′ (FIG. 2B) and MEDI522/MEDI522M (FIG. 2C) in addition an irrelevant antibody was included. The antibodies were monitored by surface plasmon resonance detection using a BIAcore 3000 instrument (Pharmacia Biosensor, Uppsala, Sweden). EphA2-Fc and αvβ3 were coupled to the dextran matrix of a CM5 sensor chip (Pharmacia Biosensor) using an Amine Coupling Kit as described (Johnsson et al., 1991, Anal. Biochem. 198:268-77 ) at a surface density of 4539 RU and 4995 RU for EphA2-Fc in FIGS. 2A and 2B respectively. αvβ3 was couple at a surface density of 4497 RU (FIG. 2C). 250 μl of each transfection supernatant (2^{nd} transfection, 2^{nd} harvest for those in FIG. 2A, 2^{nd} transfection, 1^{st} harvest for those in FIGS. 2B-C ) was injected over there respective surfaces. All binding experiments were performed at 25° C. at a flow rate of 75 μL/min; data were collected for approximately 20 min and one 1-min pulse of 1M NaCl, 50 mM NaOH was used to regenerate the surfaces. The binding data for all the antibodies is shown in FIGS. 2A-2C.

Kinetic Analysis via BIAcore:

The interaction of soluble 12G3, 4C10, 12G3/Mut and 4C10/Mut with immobilized EphA2-Fc was monitored by surface plasmon resonance detection using a BIAcore 3000 instrument (Pharmacia Biosensor, Uppsala, Sweden). EphA2-Fc was coupled to the dextran matrix of a CM5 sensor chip (Pharmacia Biosensor) using an Amine Coupling Kit as described (Johnsson et al. supra) at a surface density of 162 RU. IgGs were diluted in 0.01 M HEPES pH 7.4 containing 0.15 M NaCl, 3 mM EDTA and 0.005% P20. All subsequent dilutions were made in the same buffer. All binding experiments were performed at 25° C. with IgG concentrations typically ranging from 100 nM to 0.2 nM at a flow rate of 75 μL/min; data were collected for approximately 20 min and one 1-min pulse of 1M NaCl, 50 mM NaOH was used to regenerate the surfaces. IgGs were also flowed over an uncoated cell and the sensorgrams from these blank runs subtracted from those obtained with EphA2-Fc-coupled chips. Data were fitted to a 1:1 Langmuir binding model. This algorithm calculates both the kₒₙ and the k_{off}, from which the apparent equilibrium dissociation constant, K_{D}, is deduced as the ratio of the two rate constants (k_{off}/kₒₙ). The data are presented in Table 4.

TABLE 4
Binding Affinities

| Modified | kₒₙ | k_{off} | K_{D} |
|---|---|---|---|
| Antibody | M⁻¹ · s⁻¹ | s⁻¹ | (pM) |
| 4C10 | 1.02 × 10⁵ | 9.75 × 10⁻⁶ | 95 |
| 4C10/M | 6.41 × 10⁴ | 5.96 × 10⁻⁶ | 93 |
| 12G3 | 2.46 × 10⁵ | 8.49 × 10⁻⁶ | 34 |
| 12G3/M | 1.87 × 10⁵ | <5.0 × 10^{-6a} | <27^{a} |

^{a}Below the limit of detection

Discussion

Despite advances in recombinant antibody engineering and production, expression levels of a given antibody are often disappointing. A reproducible method to increase the producibility of any antibody by directly modifying the amino acid sequence of antibody heavy chain would be of significant benefit for the production of numerous therapeutic antibodies.

We have demonstrated for the first time that the specific substitution of one-three heavy chain residues results in a dramatic increase in the producibility of the antibody leading to improved production yields. Surprisingly, these same three substitutions reproducibly improved the producibility of seven different antibodies indicating that the identity of these three heavy chain residues is important for the producibility of an antibody. In addition we show that the substitution of these heavy chain residues does not adversely alter the antigen binding of the modified antibody and can even result in improvements of the antigen binding characteristics. Furthermore, we show that the presence of certain preferred amino acid residues at positions H40, H60 and H61 increases the producibility of antibodies containing variable domains from multiple origins including humanized and human-mouse chimeric antibodies. Taken together, these results demonstrate that the specific substitution, or specific engineering of one or more heavy chain residues at positions 40, 60 and 61 to improve the producibility of an antibody is widely applicable and can be utilized to increase the yields of virtually any antibody.

The disclosures of each and every patent, patent application, and publication cited herein are hereby incorporated by reference in their entirety for all purposes.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An antibody having at least one amino acid residue substitution, wherein the production levels of said antibody are increased compared to the antibody without said substitution.

2. The antibody of claim 1, wherein said substitution is at a residue selected from the group consisting of: 40H, 60H, and 61H, utilizing the numbering system set forth in Kabat.

3. The antibody of claim 2,
(a) wherein the amino acid residue a position 40H or 60H is substituted with alanine, or the amino acid at position 61H is replaced with aspartic acid, or
(b) wherein the amino acid residue at positions 40H and 60H are both substituted with alanine, or
(c) wherein the amino acid residues at position 40H is substituted with alanine, and the amino acid residue at position 61H is substituted with aspartic acid, or
(d) wherein the amino acid residues at position 60H is substituted with alanine, and the amino acid residue at position 61H is substituted with aspartic acid, or
(e) wherein the amino acid residues at positions 40H and 60H are substituted with alanine, and the amino acid residue at position 61H is substituted with aspartic acid.

4. The antibody of claim 1, wherein the production levels are increased by at least 1.5 fold.

5. The antibody of claim 1, wherein the production levels are increased by at least 1.5-25 fold.

6. The antibody of claim 1, wherein one or more antigen binding characteristics of said antibody are improved by at least 1%-25%.

7. The antibody of claim 1, wherein one or more antigen binding characteristics of said antibody are improved by at least 25%-100%.

8. The antibody of claim 1, wherein there is no change in any antigen binding characteristic of said antibody.

9. The antibody of claim 1, wherein there is a reduction in antigen binding of said antibody of less then 5%.

10. The antibody of claim 1, wherein there is a reduction in one or more antigen binding characteristics of said antibody of less then 5%-60%.

11. A method of producing the antibody of claim 1.

12. The method of claim 11, wherein said method comprises the steps of:
(a) substituting one or more of the amino acid residues from the group consisting of: positions 40H, 60H, or 61H, utilizing the numbering system set forth in Kabat, with alanine, alanine and aspartic acid respectively, or a conservative substitution thereof; and
(b) cultivating the host cell under conditions wherein the resulting substituted antibody is expressed by said host cell.

13. A method of increasing the production of an antibody from a eukaryotic host by at least 1.5 fold, wherein said method comprises the steps of:
(a) substituting one or more of the amino acid residues selected from the group consisting of: positions 40H, 60H, and 61H, utilizing the numbering system set forth in Kabat, with alanine, alanine and aspartic acid respectively, or a conservative substitution thereof; and
(b) cultivating the host cell under conditions wherein the resulting substituted antibody is expressed by said host cell.

14. The method of claim 13, wherein position 40H is substituted with alanine.

15. The method of claim 13, wherein position 60H is substituted with alanine.

16. The method of claim 13, wherein position 41H is substituted with aspartic acid.

17. The method of claim 13, wherein positions 40H and 60H are each substituted with alanine.

18. The method of claim 13, wherein position 40H and 61H are substituted with alanine and aspartic acid respectively.

19. The method of claim 13, wherein position 60H and 61H are substituted with alanine and aspartic acid respectively.

20. The method of claim 13, wherein position 40H, 60 and 61H are substituted with alanine, alanine and aspartic acid respectively.

21. The method of claim 13, wherein antibody production is increased by at least 1.5 to 15 fold.

22. The method of claim 13, wherein antibody production is increased by at least 15 to 50 fold.

23. The method of claim 13, wherein one or more antigen binding characteristics of said substituted antibody are improved by at least 1%-25%.

24. The method of claim 13, wherein one or more antigen binding characteristics of said substituted antibody are improved by at least 25%-100%.

25. The method of claim 13, wherein there is no change in any antigen binding characteristic of said substituted antibody.

26. The method of claim 13, wherein there is a reduction in antigen binding of said substituted antibody of less then 5%.

27. The method of claim 13, wherein there is a reduction in one or more antigen binding characteristics of said substituted antibody of less then 5%-60%.

28. The method of claim 13, wherein said host cell is a mammalian cell.

29. The method of claim 27, wherein said host cell is selected from the group consisting of:
(a) HEK293 cell,
(b) NS0 cell,
(c) CHO cell,
(d) COS cell,
(e) SP/20 cell, and
(f) PERC6 cell.

30. A substituted antibody produced by the method of claim 13.

31. The antibody produced by the method of claim 13, wherein the light chain variable region comprises any one of the amino acid sequences of SEQ ID NOS: 1-6 and the heavy chain variable region comprises any one of the amino acid sequences of SEQ ID NOS: 14-19.
